(19) United States
(12) Patent Application Publication
(10) Pub. No.: US 2010/0234756 A1
(43) Pub. Date: Sep. 16, 2010

(54) HUMAN BODY COMMUNICATION APPARATUS

(75) Inventors: Chang Hee Hyoung, Daejeon (KR); Sung Weon Kang, Daejeon (KR); Jung Hwan Hwang, Daejeon (KR); Duck Gun Park, Daejeon (KR); Sung Eun Kim, Seoul (KR); Jin Kyung Kim, Daejeon (KR); Tae Wook Kang, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/679,466

(22) PCT Filed: Sep. 17, 2008
(86) PCT No.: PCT/KR2008/005502

§ 371 Date: Mar. 22, 2010

(30) Foreign Application Priority Data
Sep. 21, 2007 (KR) 1020070096881

(51) Int. Cl. A61B 5/03; G08B 23/00

(52) U.S. Cl. 600/547; 340/573.1

(57) ABSTRACT

There is provided a human body communication apparatus capable of performing communications through the contact with external devices (a central processing unit) and a human body. The human body communication apparatus includes a plurality of contact sensor units including a plurality of electrode contacted with a human body and having conductivity, and a plurality of electrostatic sensor units coupled to the electrodes to sense an electrostatic capacity changed through the contact with the human body and generate a contact signal using the sensed electrostatic capacity; a signal analyzer unit for analyzing the contact signal received from the contact sensor units; and a control signal generator unit for generating a control signal as the analysis result in the signal analyzer unit. Therefore, the human body communication apparatus may be useful to minimize the power consumption of modem for human body communication or transceiver circuits by recognizing predetermined patterns of the user so as to reduce the unnecessary power consumption when the communication input/output apparatus, particularly a mobile device, using a human body as a medium is in contact with the device for the purpose of other uses other than the human body communications, and to minimize the power consumption to extend the standby time of the mobile devices.

### TECHNICAL FIELD

The present invention relates to a human body communication apparatus that is in contact with a human body to conduct a communication, and more particularly, to a human body communication apparatus that senses the contact with or proximity to a human body to operate a controller and a transceiver module, both of which are used for human body communication and are in a standby mode, into a normal state.

The present invention was supported by the IT R&D program of MIC/IITA[2006-S-069-03, Development of Wearable System Using Physiological Signal Processing].

### BACKGROUND ART

A communication input/output apparatus which senses the contact of the human body may apply to mobile devices such as hand phones, MP3 players, digital cameras, etc., and includes a plurality of electrodes and contact sensor units for sensing the human body contacts. When a human body approaches the mobile devices for the purpose of the basic handling of the mobile device, i.e., telecommunications, operations of numeral buttons, photography, etc., power consumption of its battery increases since the contact sensor unit senses the approach of the human body to perform unnecessary human body communication functions although a user does not want to perform the human body communication function.

For the communication system in which a human body is used as a communication medium, communication channels are formed through the contact with the human body for a period where the communication system is carried by or adjacent to the human body. However, since it is difficult to know when the devices with human body communication functions remain in contact with a human body, a user periodically checks whether a user's device forms a communication channel with the other user's device by checking a contact signal. In this case, the device does not recognize the contact with the human body even when the user's device does not remain in contact with the human body.

Therefore, a receiver front-end circuit for converting a faint signal received through a human body into a digital signal, and a micro controller for controlling the receiver front-end circuit should be always in an operation mode, or be periodically in an operation mode so as to cut down the power consumption.

Also in the case of the transmission unit including data to be transmitted, the micro controller used for human body communications applies an external control signal and data to a predetermined memory area. Therefore, a receiving circuit for confirming whether communication channels are formed, as well as a transmission circuit, should continue to operate normally. Here, although the transmission circuit starts to operate, the transmission circuit has a function to convert a digital signal received from the micro controller into a signal for applying to the human body before the transmission circuit is in contact with the human body. That is, the transmission circuit and the receiving circuit are operated to confirm whether the communication channels are formed even when there is no contact with the human body. Therefore, the power is consumed unnecessarily in the conventional devices using human body communications.

As described above, the conventional human body communication apparatuses has a problem that the transmission and receiving circuits and the micro controller for controlling the transmission and receiving circuits should be always or periodically in a normal operation mode even when the transmission and receiving circuits are not in contact with a human body, the transmission and receiving circuits should operate to confirm whether communication channels are formed. This indicates that power is unnecessarily consumed in the conventional human body communication apparatuses. Also, the communication input/output apparatus that may sense simple human body contacts has a problem that the unnecessary power consumption is caused in modems and transceiver modules for human body communication since it is impossible to interpret the exact intention of a user when the user approaches the device for the purpose of other uses other than the human body communications.

When there is no contact of a user with the human body communication apparatus having a contact sensor unit, the human body communication apparatus may reduce its consumption of electric power, which is used to judge whether the communication channels are formed, by applying the human body communication apparatus with a contact sensor unit to portable devices such as a hand phone. However, when a human body approaches and contacts with electrodes of the devices for the purpose of other operations, for example contacts for other uses of a user such as communications, listening to music, or taking photographs, these human body communication apparatuses sense the contact and approach to the human body, regardless of the intentions of a user, to generate a control signal. Therefore, there is a need to prevent the unnecessary power consumption since the human body communication apparatuses transmit/receive data to/from the external devices using the control signal.

### DISCLOSURE OF INVENTION

### Technical Problem

The present invention is designed to solve the problems of the prior art, and therefore it is an object of the present invention to provide a human body communication apparatus capable of reducing the unnecessary power consumption of transmission/reception units and external devices that may be caused before the contact with a user's human body when the device with human body communication unit communicates with other devices using user's body as a medium, and of reducing the unnecessary power consumption that may be caused through the contact with the devices for the purpose of other uses other than the human body communications.

### Technical Solution

According to an aspect of the present invention, there is provided a human body communication apparatus includes a plurality of contact sensor units including a plurality of electrode contacted with a human body and having conductivity, and a plurality of electrostatic sensor units coupled to the electrodes to sense an electrostatic capacity changed through the contact with the human body and generate a contact signal using the sensed electrostatic capacity; a signal analyzer unit for analyzing the contact signal received from the contact sensor units; and a control signal generator unit for generating a control signal as the analysis result in the signal analyzer unit.

### ADVANTAGEOUS EFFECTS

As described above, the communication input/output apparatus according to the present invention may be useful to minimize the power consumption of modem or transceiver circuits for human body communication by sensing the certain changes in electrostatic capacity by the contact of a user using a plurality of electrodes and an electrostatic sensor unit to recognize predetermined patterns of the user so as to reduce the unnecessary power consumption caused by the simple contacts, and to minimize the power consumption to extend the standby time of mobile devices.

Also, the communication input/output apparatus according to the present invention may be useful to apply to electrostatic switches, which are used in a hand phone and an MP3 player, without any change in the appearance of the applied devices since the electrostatic switches have excellent durability when compared to physical switches, and to enhance the convenience of users by employing user's operation functions that are suitable for various mobile devices and also applying the same patterns to the mobile devices.

### BRIEF DESCRIPTION OF THE DRAWINGS

FIG. 1 is a unit view illustrating a configuration of a human body communication apparatus including a plurality of electrodes and an electrostatic sensor unit according to exemplary embodiments of the present invention.

FIG. 2 is a unit view illustrating a configuration of a human body communication apparatus using a time division duplexer including a human body contact sensor unit according one exemplary embodiment of the present invention.

FIG. 3 is a unit view illustrating a configuration of a human body communication input/output apparatus using a frequency division duplexer including a human body contact sensor unit according another exemplary embodiment of the present invention.

FIG. 4 is a unit view illustrating a configuration of a human body communication input/output apparatus using an electric field sensor according still another exemplary embodiment of the present invention.

### BEST MODE FOR CARRYING OUT THE INVENTION

Hereinafter, exemplary embodiments of the present invention will now be described in detail with reference to the accompanying drawings, for the purpose of better understanding of the present invention as apparent to those skilled in the art. For the detailed description of the present invention, it is however considered that descriptions of known components and their related configurations according to the exemplary embodiments of the present invention may be omitted since they are judged to make the gist of the present invention unclear.

The exemplary embodiment of the present invention is related to a human body communication apparatus using the human body contact sensor unit in which a large amount of electrodes and an electrostatic sensor unit are used to sense the certain changes in electrostatic capacity due to the contact of a user. Hereinafter, the human body communication apparatus according to the exemplary embodiments of the present invention will be described in more detail with reference to the accompanying drawing.

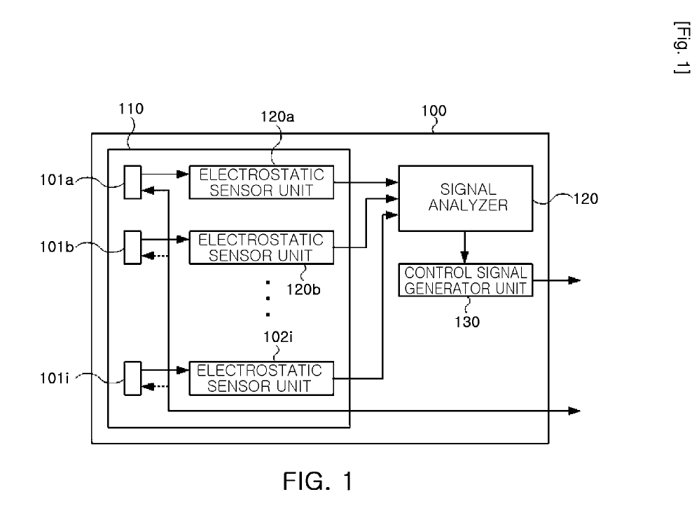

FIG. 1 is a unit view illustrating a configuration of a human body communication apparatus using the human body contact sensing according to exemplary embodiments of the present invention.

Referring to FIG. 1, the human body communication apparatus 100 may include a contact sensor unit 110, a signal analyzer unit 120, and a control signal generator unit 130.

The contact sensor unit 110 includes a plurality of electrodes 101a to 101i giving/taking a signal to/from a human body, and a plurality of electrostatic sensor units 102a to 102i functioning to detect an electrostatic capacity generated by the contact and proximity. Here, at least one electrode out of a plurality of the electrodes is coupled to a transmission/reception unit or modem for human body communications, and at least one electrode coupled to the transmission/reception unit or modem forms a continuous contact point with the human body during a period of human body communications.

The signal analyzer unit 120 analyzes an electrical signal outputted from a plurality of the electrostatic sensor units 102a to 102i of the contact sensor unit 110.

The control signal generator unit 130 receives the signals analyzed by the signal analyzer unit 120 to generate a control signal.

The human body communication apparatus as configured thus couples signals between at least one electrode of the electrodes 101a to 101i that are in contact with the human body and the transmission/reception unit or modem for human body communications when the certain changed patterns in given electrostatic capacity are generated by a user. In this procedure, it is possible to improve standby time characteristics of the mobile devices used for the human body communication by minimizing its power consumed due to the erroneous operations when a user is in contact with and approaches the devices for the purpose of other uses rather than the human body communication.

The human body communication apparatus according to one exemplary embodiment of the present invention, as configured thus, will be described in detail with reference to the accompanying drawing. Here, the human body communication apparatus according to one exemplary embodiment of the present invention is a human body communication apparatus having a time division duplex system.

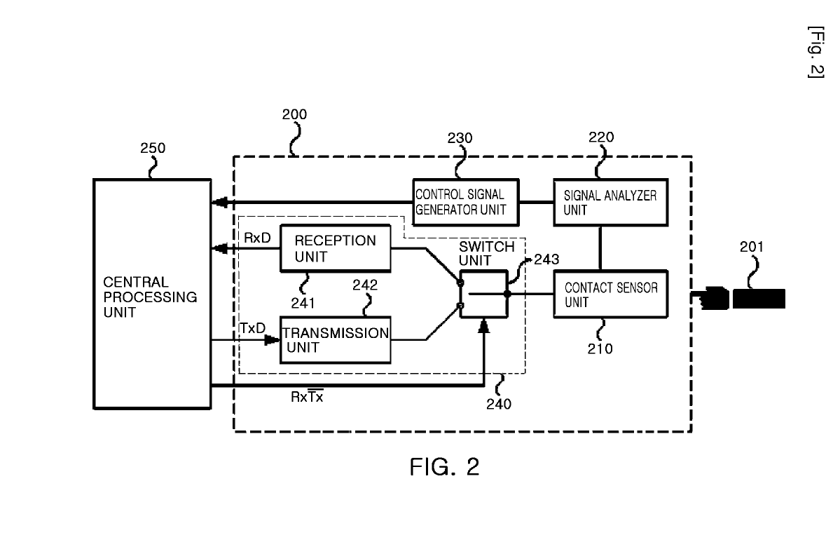

FIG. 2 is a unit view illustrating a configuration of a human body communication apparatus using time division duplex system including a human body contact sensor unit according to one exemplary embodiment of the present invention.

Referring to the FIG. 2, the human body communication apparatus 200 having a time division duplex system is usable in the use of same carrier frequencies both transmit frequency and receive frequency, and includes a contact sensor unit 210, a signal analyzer unit 220 and a control signal generator unit 230, as shown in FIG. 1. Here, the human body communication apparatus 200 may further include a data signal processor unit 240 having a time division multiplex system.

Here, the data signal processor unit 240 may be composed of a reception unit 241, a transmission unit 242 and a switch unit 243. In this case, the reception unit 241 has a continuous frequency demodulation means, and the transmission unit 242 includes a continuous frequency modulation means. The switch unit 243 selects the transmission of a data signal to the human body 201 or the reception of a data signal from the human body 201 according to the control signal generated from the control signal generator unit 230. Here, the baseband signal includes a UWB signal having a very short pulse cycle as a spreading and channel-encoded signal in which characteristics of the communication channels are reflected.

The control signal generator unit 230, the reception unit 241 and the transmission unit 242 are coupled to a central processing unit (CPU) 250.

The contact sensor unit 210 includes a plurality of electrodes and a plurality of electrostatic sensor units.

Then, an operation of the human body communication apparatus having a time division multiplex system, as configured thus, will be described in detail.

The contact sensor unit 210 senses an electrostatic capacity (or a magnetic field) changed through the contact with the human body 201 to generate an electrical signal, and transmits the generated electrical signal to the signal analyzer unit 220. Then, the signal analyzer unit 220 analyzes a signal as a user's intention from an electrical signal pattern generated by the change in electrostatic capacity. The analyzed signal is inputted into the control signal generator unit 230. As a result, the control signal generator unit 230 confirms the analyzed signal, and then generates a wake-up signal as a control signal in the central processing unit 250. The output of the control signal generator unit 230 makes it possible to apply a signal so that the central processing unit 250 can normally operate in a standby mode, and also controls a power supply of the data signal processor unit 240.

The central processing unit 250, which operates with the reception of the control signal, transmits a transmitted data signal (TxD) to the transmission unit 242. Therefore, the transmission unit 242 converts the transmitted data signal (TxD) into a baseband signal, and transmits the converted baseband signal to the contact sensor unit 210 through the switch unit 243. Then, the contact sensor unit 210 applies the baseband signal to the human body 201 that is in contact with at least one electrode.

In this case, the contact sensor unit 210 senses an electrostatic capacity that is changed through the contact with human body coupled to the electrode, and transmits the sensed electrical signal as a baseband signal to the reception unit 241 through the switch unit 230. Therefore, the reception unit 241 converts the transmitted baseband signal into a received data signal (RxD) as a digital signal, and transmits the converted digital signal to the central processing unit 250.

Subsequently, the above-mentioned human body communication apparatus according to another exemplary embodiment of the present invention will be described in more detail with reference to the accompanying drawing. As a human body communication apparatus having a time division duplex system using a human body contact sensor unit in the use of different carrier frequencies such as transmit frequency and receive frequency, the human body communication apparatus according to another exemplary embodiment of the present invention will be described in detail with reference to the accompanying drawing.

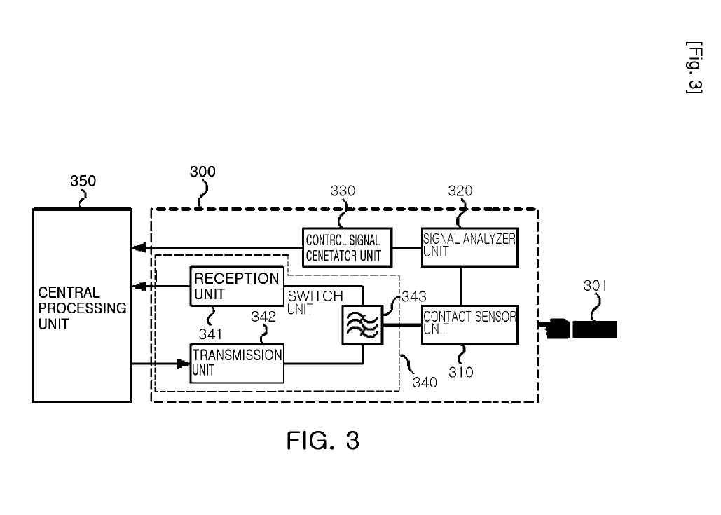

FIG. 3 is a unit view illustrating a configuration of a frequency-divided human body communication input/output apparatus having a human body contact sensor unit according to another exemplary embodiment of the present invention.

Referring to FIG. 3, the human body communication apparatus 300 with a frequency division system includes a duplexer 343 for dividing paths of two signals in the use of the different carrier frequencies such as transmit frequency and receive frequency, and also may include a contact sensor unit 310, a signal analyzer unit 320, a control signal generator unit 330, and a data signal processor unit 340. Here, the data signal processor unit 340 includes a reception unit 341 and a transmission unit 342 and a duplexer 343.

Then, an operation of the human body communication apparatus with a frequency division system, as configured thus, will be described in detail.

The contact sensor unit 310 senses an electrostatic capacity (or a magnetic field) that is changed through the contact with the human body 301 to generate an electrical signal, and transmits the generated electrical signal to the signal analyzer unit 320. Then, the signal analyzer unit 320 analyzes a signal as a user's intention from an electrical signal pattern generated by the change in electrostatic capacity. The analyzed signal is inputted into the control signal generator unit 330. As a result, the control signal generator unit 330 confirms the analyzed signal, and then generates a wake-up signal as a control signal in the central processing unit 350. The output of the control signal generator unit 330 makes it possible to apply a signal so that the central processing unit 350 can normally operate in a standby mode, and also controls a power supply of the data signal processor unit 340. Therefore, the central processing unit 350 operates according to the control signal to transmit a transmitted data signal (TxD) as a digital signal to the transmission unit 342.

Therefore, the transmission unit 342 converts the received transmitted data signal (TxD) into a baseband signal, subjects the converted baseband signal to a continuous frequency modulation process using a carrier frequency, and transmits the baseband signal to the contact sensor unit 310 through the duplexer 343. Therefore, the contact sensor unit 310 applies the received signal to the human body 301. Unlike as shown in FIG. 2, a selection signal from the central processing unit 350 is required in this case.

In this case, the contact sensor unit 310 senses an electrical signal from the human body 301 that is in contact with internal electrodes, converts the sensed electrical signal into a received data signal (RxD) as a digital signal, and transmits the converted digital signal to the reception unit 341 through the duplexer 343. Therefore, the reception unit 341 restores the signal, which is received through the human body 301, into a received data (RxD) as a digital signal by using a continuous frequency demodulation circuit, and transmits the restored digital signal to the central processing unit 350.

Examples of the signals that may communicate using the above-mentioned human body communication apparatus include a pulse-modulated signal and a continuous frequency modulated signal, all of which include Manchester coding. A filter may be used in an output end to enhance the frequency efficiency and limit the spurious frequency in the use of the pulse-modulated signals.

The human body communication apparatus according to the present invention transmits and receives using a digital signal by means of the time division duplex system without any of the continuous frequency modulation of the digital signal. In this case, the reception unit 341 is composed of circuits for restoring the inputted faint signal into a digital signal, and the transmission unit 342 may have an amplification function as a circuit for converting a signal received from the central processing unit 350 into a receivable signal.

Meanwhile, the human body communication apparatus according to still another exemplary embodiment of the present invention, which may detect an electric field induced from the human body in a non-contact state using an electro-optic effect, will be described in detail with reference to the accompanying drawing.

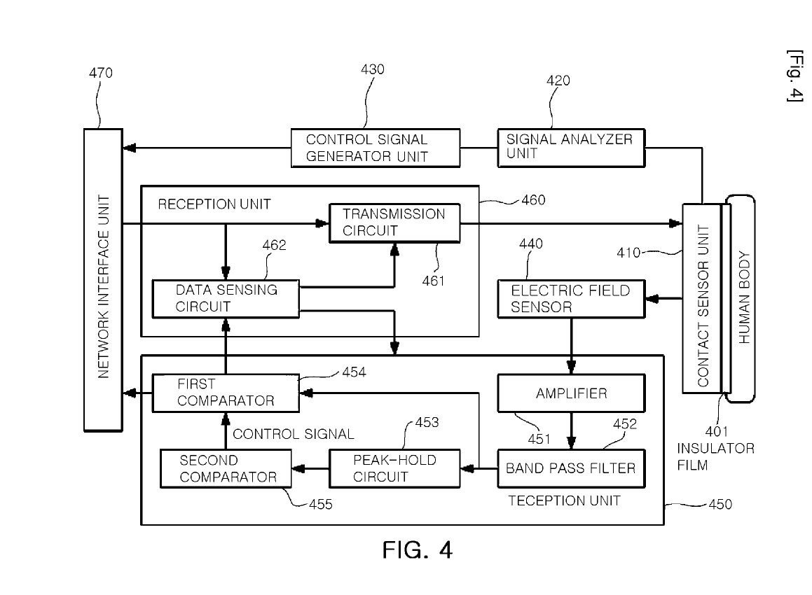

FIG. 4 is a unit view illustrating a configuration of a human body communication apparatus using an electric field sensor according to still another exemplary embodiment of the present invention.

Referring to FIG. 4, the human body communication apparatus according to still another exemplary embodiment of the present invention may include a contact sensor unit 410 in which an insulator film 401 is attached to a region that is in contact with a human body, a signal analyzer unit 420, a control signal generator unit 430, an electric field sensor 440, a reception unit 450, a transmission unit 460, a network interface unit 470.

The contact sensor unit 410 includes contact pads having conductivity, for example a transceiver electrode and an electrostatic sensor unit. Here, the electric field sensor 440, the reception unit 450 and the transmission unit 460, all of which have high power consumption prior to the human body communication, may cut down their power consumption by analyzing certain patterns of a user for the human body communications.

The reception unit 450 includes an amplifier 451 for amplifying a signal received from the electric field sensor 440, a band pass filter 452 for filtering the amplified signal, a peak-hold circuit 453 and first and second comparators 454 and 455.

The transmission unit 460 includes a transmission circuit 461 for generating a transmitting signal, and a data sensing circuit 462 for transmitting a control signal to the transmission circuit 461 and the reception unit 450.

Here, it is considered that specific functions of the transmission unit 460 and the reception unit 450 may be understood by those skilled in the art, and their detailed descriptions are omitted for clarity.

Then, an operation of the human body communication apparatus according to still another exemplary embodiment of the present invention will be described in detail.

The transmission unit 460 transmits a received signal inputted from the network interface unit 470 to the contact sensor unit 410. Therefore, the contact sensor unit 410 applies the received signal to a human body through an insulator film 401.

Subsequently, the contact sensor unit 410 senses a faint electric field through the insulator film 401 in the human body with it being in non-contact with the human body, and then transmits the sensed electric field signal to the signal analyzer unit 420 and the electric field sensor 440. Therefore, the signal analyzer unit 420 analyzes the electric field signal received from the contact sensor unit 410, and transmits the analyzed electric field signal to the control signal generator unit 430. Then, the control signal generator unit 430 confirms the analyzed signal to generate a control signal in the network interface unit 470.

In this case, the electric field sensor 440 converts the faint electric field signal inputted from the contact sensor unit 410 into an electrical signal, and then transmits the converted electrical signal to the reception unit 450. Therefore, the reception unit 450 transmits the received electrical signal to the network interface unit 470.

According to the exemplary embodiments of the present invention as described above, an operation principle of a contact sensor constituting the contact sensor units is that a natural oscillation frequency is generated by the reference electrostatic capacity when there is no contact with the human body and a load electrostatic capacity is changed when there in contact with the human body, and therefore an output signal is generated through comparators due to the changes in oscillation frequency. It is considered that the operation principle of the contact sensor may be easily understood by those skilled in the art, and therefore detailed description of the operation principle omitted for clarity.

The electrodes used in the present invention may be composed of conductive materials to sense the direct contact with a human body, and also composed of conductive materials coated with dielectrics. Also, each of the electrodes may be composed of multi-channel sensors when a plurality of transceiver electrodes are formed to maintain stable contact points with a human body.

The contact-type sensor-driven electrostatic sensor unit may control the contact sensitivity by controlling the reference electrostatic capacity, and the sensors that are set to have high contact sensitivity may sense the proximity that a distance between the contact pads (or electrodes) and the human body ranges from several millimeter (mm) to several centimeter (cm). In this case, it is possible to reduce the time, which spans from the delivery of a user's intention for human body communication to the actual communication, and also to reduce the time when a transceiver circuit comes to a normal state, by controlling the time until a control signal is generated through the signal analyzer unit.

The micro controller disclosed in the present invention may be installed inside the communication input/output apparatus, but micro controllers may be used in devices for human body communication, for example, a hand phone, PDA, an MP3 layer, a portable image information system, PC, a notebook computer, a printer, etc.

The communication input/output apparatus for sensing a human body contact according to the present invention may be used for the communication between mobile devices, the communications using a human body as a medium between the mobile devices and fixed devices and between the fixed device and fixed device, etc.

The functions of the apparatus and method disclosed in this application may be realized as computer-readable codes in computer-readable recording media. The computer-readable recording media include all kinds of recording devices in which data that are readable by a computer system are being stored. Examples of the computer-readable recording media include ROM, RAM, CD-ROM, a magnetic tape, a floppy disk, an optical data storage device, etc., and may be also realized in the form of a carrier wave (for example, transmission through the internet). In addition, the computer-readable recording media may be distributed into the computer system that is connected through the networks to store and implement the computer-readable codes in a distribution mechanism. Furthermore, functional programs, codes and code segments, all of which are used to practice the present invention may be easily deduced by programmers in the art to which the present invention belongs.

As described above, the exemplary embodiments of the present invention have been described in detail referring to the accompanying drawings. However, it should be understood that the terms used in the specification and appended claims should not be construed as limited to general and dictionary meanings, but interpreted based on the meanings and concepts corresponding to technical aspects of the present invention on the basis of the principle that the inventor is allowed to define terms appropriately for the best explanation. Therefore, the description proposed herein is just a preferable example for the purpose of illustrations only, not intended to limit the scope of the invention, so it should be understood that other equivalents and modifications could be made thereto without departing from the spirit and scope of the invention.

1. A human body communication apparatus, comprising:
a plurality of contact sensor units including a plurality of electrode contacted with a human body and having conductivity, and a plurality of electrostatic capacity sensor units coupled to the electrodes to sense an electrostatic capacity changed through the contact with the human body and generate a contact signal using the sensed electrostatic capacity;
a signal analyzer unit for analyzing the contact signal received from the contact sensor units; and
a control signal generator unit for generating a control signal as the analysis result in the signal analyzer unit.

2. The human body communication apparatus of claim 1, wherein the electrostatic sensor unit senses the direct contact between the human body and a plurality of the electrodes, or the approach of the human body.

3. The human body communication apparatus of claim 1, further comprising a data signal processor unit for transmitting/receiving a data signal to/from external devices.

4. The human body communication apparatus of claim 3, wherein the data signal processor unit transmits/receive the data signal through a time division multiplex system.

5. The human body communication apparatus of claim 3, wherein the data signal processor unit transmits/receive the data signal through a frequency division system using a frequency modulated continuous wave mode in which a transmitting carrier frequency and receiving carrier frequency are different to each other.

6. The human body communication apparatus of claim 4, wherein the data signal processor unit comprises:
a reception unit for restoring the baseband signal sensed from the human body to an original digital signal;
a transmission unit for applying the received data signal received from the external device as a baseband signal to the human body; and
a switch unit coupled to the transmission unit and the reception unit to select the transmission to the human body or the reception from the human body according to the control signal.

7. The human body communication apparatus of claim 4, wherein the data signal includes a UWB signal having a very short pulse cycle as a spreading and channel-encoded signal in which characteristics of the communication channels are reflected.

8. The human body communication apparatus of claim 4, wherein the data signal processor unit comprises:
an electric field sensor unit for converting the received data signal, in the form of an electric field signal induced from the contact sensor unit, into an electrical signal when there is no contact with the human body;
a reception unit for restoring the electrical signal received from the electric field sensor unit to an original digital signal; and
a transmission unit for applying the transmitted data signal received from the external device as a baseband signal to the human body.

9. The human body communication apparatus of claim 5, wherein the data signal processor unit comprises:
a reception unit for subjecting the received data signal received from the contact sensor unit to a continuous frequency demodulation process and restoring the received data signal to an original digital signal;
a transmission unit for subjecting the transmitted data signal, in the form of a digital signal received from the external device, to a continuous frequency modulation process and applying the transmitted data signal to the human body; and
a duplexer for dividing paths of the transmitted data signal and the received data signal.

10. The human body communication apparatus of claim 6, wherein the transmission unit processes the transmitted data signal inputted from the external device with a frequency modulated continuous wave mode using the same carrier frequency.

11. The human body communication apparatus of claim 6, wherein the reception unit processes the received data signal transmitted from the contact sensor unit with the frequency demodulated continuous wave mode.
